(19) United States
(12) Patent Application Publication
(10) Pub. No.: US 2005/0249636 A1
(43) Pub. Date: Nov. 10, 2005

(54) Bubble valve for sorting of cells and the like

(76) Inventors: Christopher Andrew Tacklind, Palo Alto, CA (US); Cameron Andras Tacklind, Palo Alto, CA (US)

Correspondence Address: Chris Tacklind, 250 Cowper Street, Palo Alto, CA, 94301, US

(21) Appl. No.: 10/838,308

(22) Filed: May 4, 2004

(51) Int. Cl. G01N 33/00

(52) U.S. Cl. 422/73; 422/100

(57) ABSTRACT

A method and apparatus are presented for a microscopic valve. The valve is electronically activated. Sensors for detecting objects in the flow may be external or formed in the channels of the valve. Many valves can be formed in parallel and in sequence on a single substrate. Multiple channels may feed each junction. Closure of the valve is accomplished by the formation of a vapor bubble or bubbles. Virtual walls may be formed by a sequence of bubbles. Logic and driver circuitry for producing bubbles may be external or included in the substrate. Such an array is ideally suited for sorting cells. Other materials in a suspension may also be sorted by a variety of criteria. A multi lumen output can produce a continuous distribution of cells or particles thus sorted.

### BACKGROUND OF THE INVENTION

The centrifuge is a time-honored method for increasing the density of particles. By effectively increasing the acceleration of gravity many fold, more dense materials and particles are readily pushed to the bottom of a test tube. This action discriminates only by the density of the particles. Yet it is effective for many suspensions of cells and other particles. For example, it is used for concentrating the density of red blood cells. Drawing off the fluids and lighter cells thus performs the crude sorting of cells in blood.

Many modern medical therapies would be possible if individual cells could be sorted by more discriminating methods. Tagging of cells with fluorescent markers and other methods make it possible to identify cells of interest. But the process of sorting the individual cells is limited to a time consuming process.

Prior art discloses suspending cells or particles in a stream of fluid. External sensing means can detect and type on the order of 10,000 cells per second. Breaking the stream into droplets captures individual cells in a droplet. A charge may be applied to the droplet. Electrostatic forces may be used to selectively deflect the droplets. Collecting the droplets in separate receptacles provides the desired sort.

Other discrimination methods may be employed such as particle size detection, optical absorption, and thermal conductivity etcetera.

### DISCUSSION OF PRIOR ART

Microscopic vapor bubbles are commonly used as an actuator in ink jet printers such as U.S. Pat. No. 4,490,728 “Thermal ink jet printer”. These use the formation of a vapor bubble to expel ink from a small channel.

U.S. Pat. No. 6,062,681 “Bubble Valve and bubble valve-based pressure regulator” describes a channel with a bubble formed in it for pressure regulation.

This is an obstruction in the tube not a diverter from one tube to another.

Thomas K. Jun of UCLA uses a series of sequenced bubbles to pump fluids through a channel in his publication “Micro Bubble Pump”.

U.S. Pat. No. 5,878,527 “Thermal optical switches for light” uses vapor bubbles to form optical switches in fiber optic junctions.

### SUMMARY OF THE INVENTION

The invention at hand is a microscopic valve. As a fluid flows through a “Y” junction, fluid is diverted to one leg or the other. This is done by momentarily closing the fluid channel of one leg or the other. The channel is closed by formation of a vapor bubble in the channel. Fluid and objects in the fluid are thus diverted to the opposite leg.

Particles of many types may be suspended in the flow. Detection means may be provided to determine a property of the fluids and particles flowing through. Detection means may be external or integrated into the substrate. Switching control may be internal or externally actuated. Switching may be in response to the properties detected.

The valves may be mass-produced in an array that processes particles through thousands of adjacent channels simultaneously. An array of such valves provides a simple integrated method for sorting cells. It is compact and scaleable to process a large volume of cells in parallel in a reasonable time.

Other applications include programmed mixing of solutions or gasses. Printing applications include mixing of ink. This can be used to alter dye or pigment density variations. Solutions and particles that are sorted can be arranged in desired orders.

### BRIEF DESCRIPTION OF THE DRAWINGS

FIG. 1 shows an oblique schematic view of a bubble actuated Y-valve with the top of the channels removed for clarity.

FIGS. 2.1 through FIG. 2.3 show a sequence of events used to direct a particle.

FIG. 3.1 through FIG. 3.4 show a sequence of events used to direct a particle with a generalized detector.

FIG. 4.1 through FIG. 4.5 show a sequence of events used to direct a particle with a specific sensor employing an integrated light source and light detector.

FIG. 5.1 and FIG. 5.2 show oblique views of two configurations of bubble actuated T-valves.

FIG. 6 shows an oblique view of a generalized X-valve.

FIG. 7 shows a plan view of an array of generalized X-valves.

FIG. 8 shows a plan view of an array of generalized T-valves.

### DETAILED DESCRIPTION OF THE INVENTION

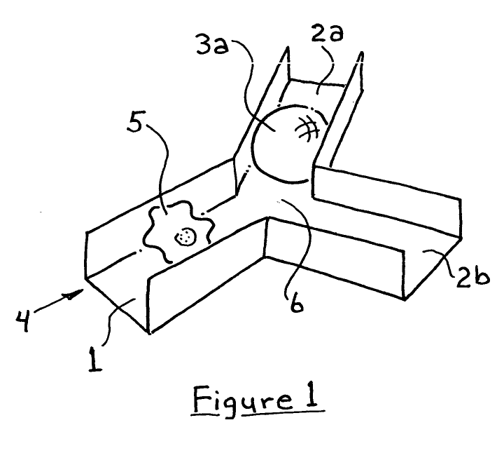

As depicted in FIG. 1, a “Y” junction is formed by an entrance channel (1) and two output channels (2a) and (2b). The paths may be of the same cross-sectional area or differing cross-sectional area. A working fluid (4) is allowed or forced to flow from the entrance channel to the exit channels. The fluid is roughly divided between the two exit channels. Any particles (5) in the fluid will approach the junction (6). The particles will randomly go to one exit channel or the other exit channel. A vapor bubble (7a) is shown in the mouth of channel 2a.

The fluid may be externally pumped into entrance channels or pulled from exit channels by methods common in the art. These include but are not limited to mechanical pumps, peristaltic pumps, gravity feed etcetera. Pumping means may be included on the substrate. Pumping means may be a sequence of bubbles. Pulses in the pumped stream may be synchronized with the valving functions.

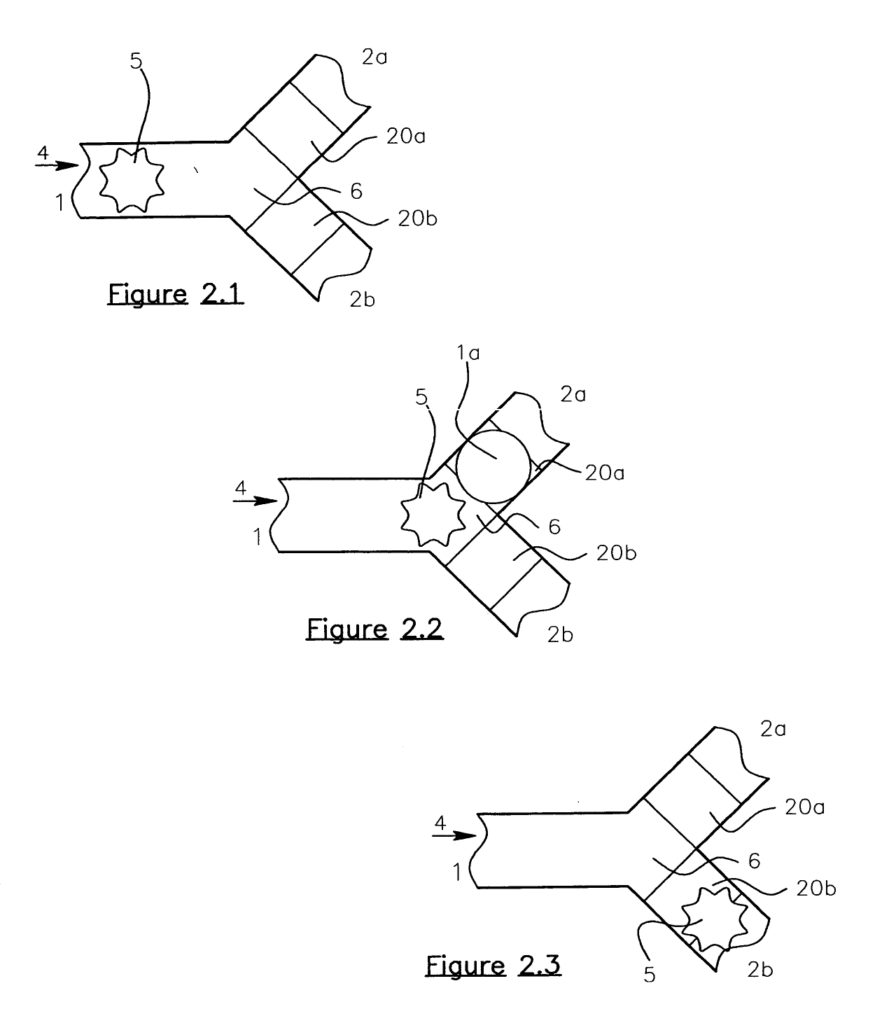

FIG. 2.1 through FIG. 2.3 show the basic sequence of steps of a valve function. A working fluid (4) is pumped into the entrance channel (1). The working fluid may be water, aqueous solution, or any other fluid with a vapor point and viscosity suitable for the particular application. If a bubble (7a) is formed in the mouth of channel (2a) the flow will be directed to exit channel (2b). Alternately a bubble (7b) may be formed in channel (2b). This will direct the flow to exit channel (2a).

FIG. 2.1 shows a particle or particles (5) suspended and carried along within the working fluid (4). As the particle approaches the junction (6) a vapor bubble (7a) is formed in the mouth of output channel (2a) as shown in FIG. 2.2. This restricts the flow through channel (2a). The fluid and particle are carried along into exit channel (2b) as shown in FIG. 2.3. Alternately, a bubble may be formed in the mouth of channel (2b) directing the flow to exit channel (2a).

Bubbles may be formed by external means. This includes, but is not limited to, an external laser. The laser may be directed to form a bubble inside the working fluid. Alternately, energy dissipating features (3a) and (3b) may be included at the mouths of channel (2a) and (2b). Laser energy may be directed at these features. External light may be used to trigger a light activated switch. The substrate may be temperature controlled to a desired point near the boiling point of the working fluid (4). A super heated fluid can be triggered to nucleate by external energy source directed at the bubble generating site.

The energy dissipating features (3a) and (3b) may be thin film resistors. A current pulse may be passed through either of the thin film resistors. The heat dissipated in the resistor is coupled to the fluid in contact with the resistor. Vaporization of the thin layer occurs and a bubble is produced. The bubble may be sustained by energy dissipation. Once the heating ceases, the vapor quickly condenses and the bubble collapses. Various pulse widths and pulse shapes may be employed.

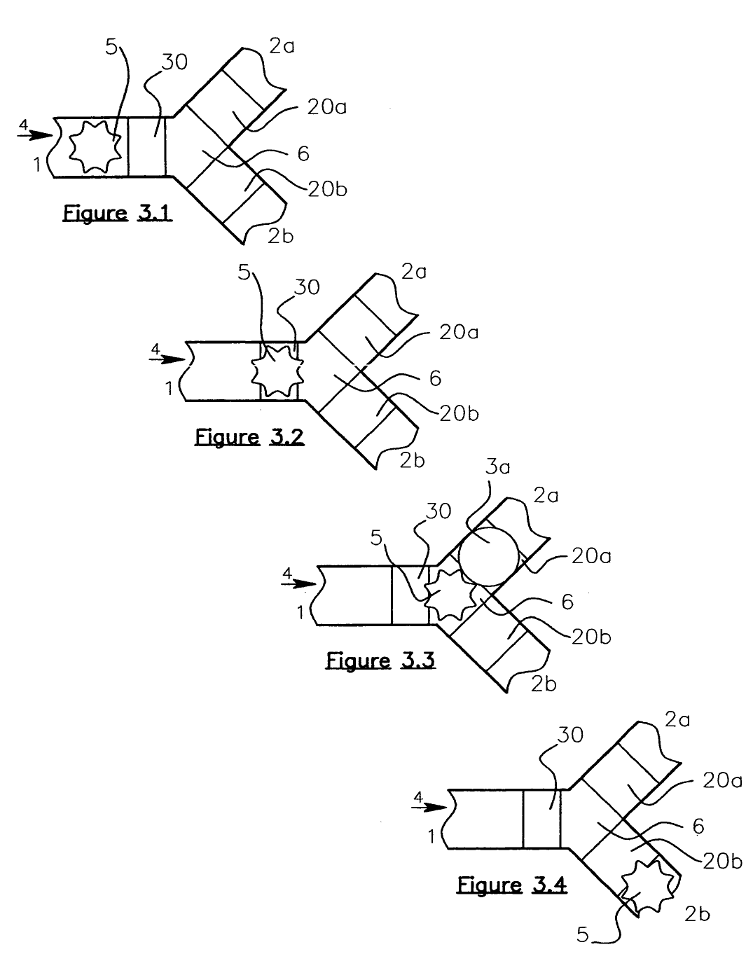

FIG. 3.1 through FIG. 3.4 show the basic operation of the valve used for sorting. FIG. 3.1 shows the working fluid (4) carrying along with it an occasional particle (5). The working fluid flows roughly equally through exit channels (2a) and (2b). In FIG. 3.2 the particle passes over detector (30). The detector may be built into the channel or be an external device. The detector may be suited to detect any desired property of the fluid or particle. If the property is found, an actuation means causes a bubble (3a) to be formed when the particle reaches the junction (6) as seen in FIG. 3.3. This causes the flow and the particle to be diverted to exit channel (2b) as seen in FIG. 3.4. Alternately, if the desired property is not found, a bubble could be formed at the mouth of exit channel (2b) causing the flow and the particle to de diverted to exit channel (2a).

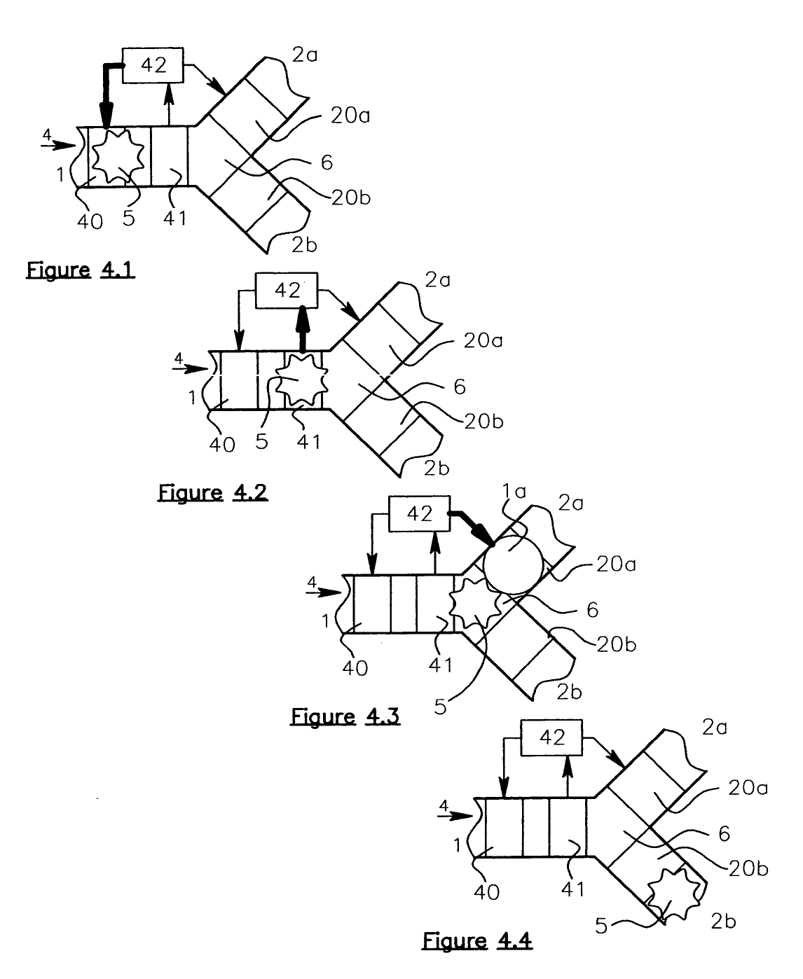

FIG. 4.1 through FIG. 4.5 show one embodiment of a sensor in operation. This example, in no way restricts the generality of sequences that may be employed. In FIG. 4.1 a particle (5) is carried in the flow (4). The particle includes a fluorescent dye. In FIG. 4.2 the particle passes over a light emitting diode (40) formed in the entry channel (1). The photons excite the fluorescent dye on the particle (5). In FIG. 4.3 the working fluid (4) brings the particle past a light detector (41). In this case the rate of emitted photons is detected. As seen in FIG. 4.4 a logic and driver circuit (42) causes thin film resistor (3a) in exit channel (2a) to be energized. This causes the particle to pass to exit channel (2b) as seen in FIG. 4.5.

Sensors may be made to detect a wide variety of properties as are known in the art. These include but are not limited to particle size, shadow cast, spectroscopy, emissivity, absorption, fluoresce, density, thermal conductivity, radioactivity, radioactive decay rate, etcetera. Chemical sensors can also detect toxins.

Radioactive particles are also readily detected. Particles may be irradiated and be rendered temporarily radioactive. The amount of radiation is readily detected and can be used as a criterion for sorting. The time decay of the radioactivity can also be used as an indicator. If the radiological properties of the particles in a suspension are cataloged, then the sorting can be used to identify the quantity of each constituent in the suspension.

Thermal properties can be exploited also. Heat pulses in the flow may be used to track the velocity of the fluid. Heat decay rates can be detected and used for categorizing materials.

The detector sites can also be used as chemistry sites. External means or catalysts at the site can cause chemical reactions to occur. The reactants may be detected. The bubble or bubbles can be used to delay the fluid flow to allow the needed time for the chemical reaction or time for detection.

Detector sites may be used to trigger a bubble while a strand is traversing a bubble generation site. The bubble formation may cleave the strand. Strands may be directed by subsequent channels and valves to be reconstructed at later sites.

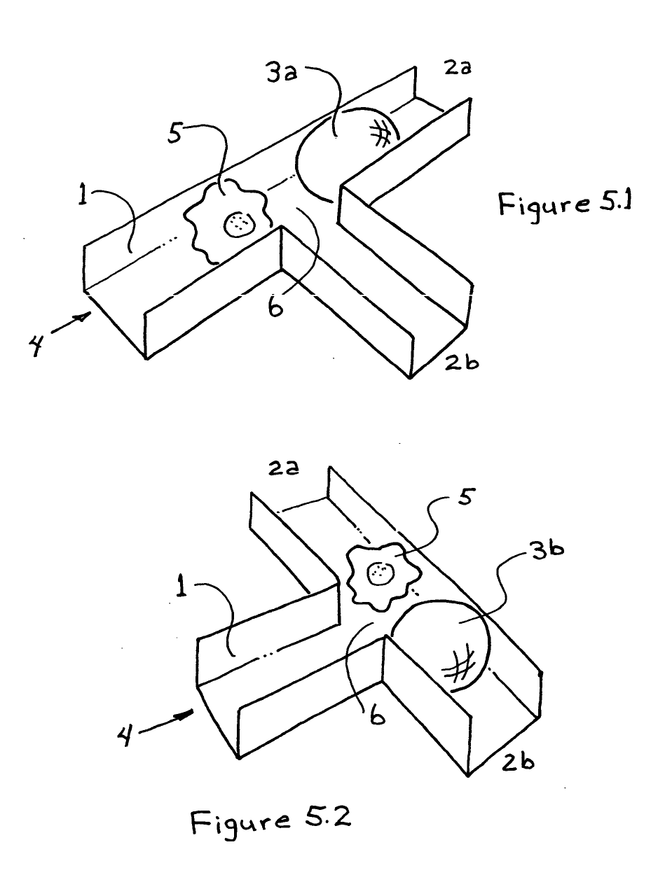

Other valve configurations are possible. A “T” shaped junction can be employed. Without loss of generality, two examples are shown in FIG. 5.1 and FIG. 5.2.

A natural extension of this sorting process is to make the sorting decisions in a widely parallel array. While this is possible with Y-valves or T-valves, these configurations lead to ever increasing density of channels. A hexagonal array eliminates this problem but is not favorable for production in silicon.

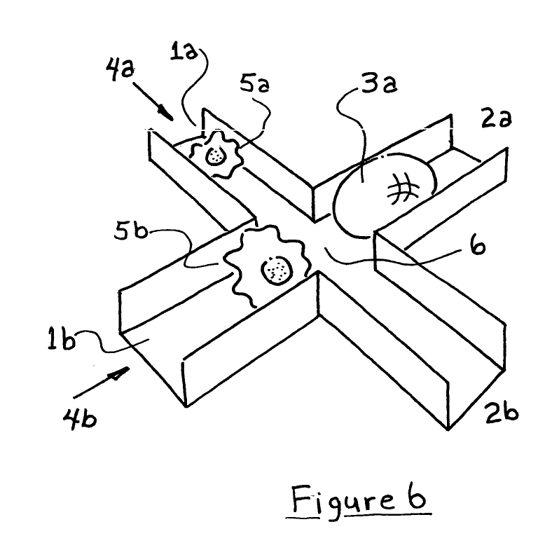

As seen in FIG. 6 another useful configuration is an “X” or “+”. Such an X-valve has two entrance channels (1a) and (1b) which feed to two exit channels (2a) and (2b). Sensors may be disposed at one or both inputs. Bubble sites may be in one or both of the exit channels. X-valves more readily allow the concatenation of valves. Channels can be readily fabricated using an-isotropic etching of silicon wafers.

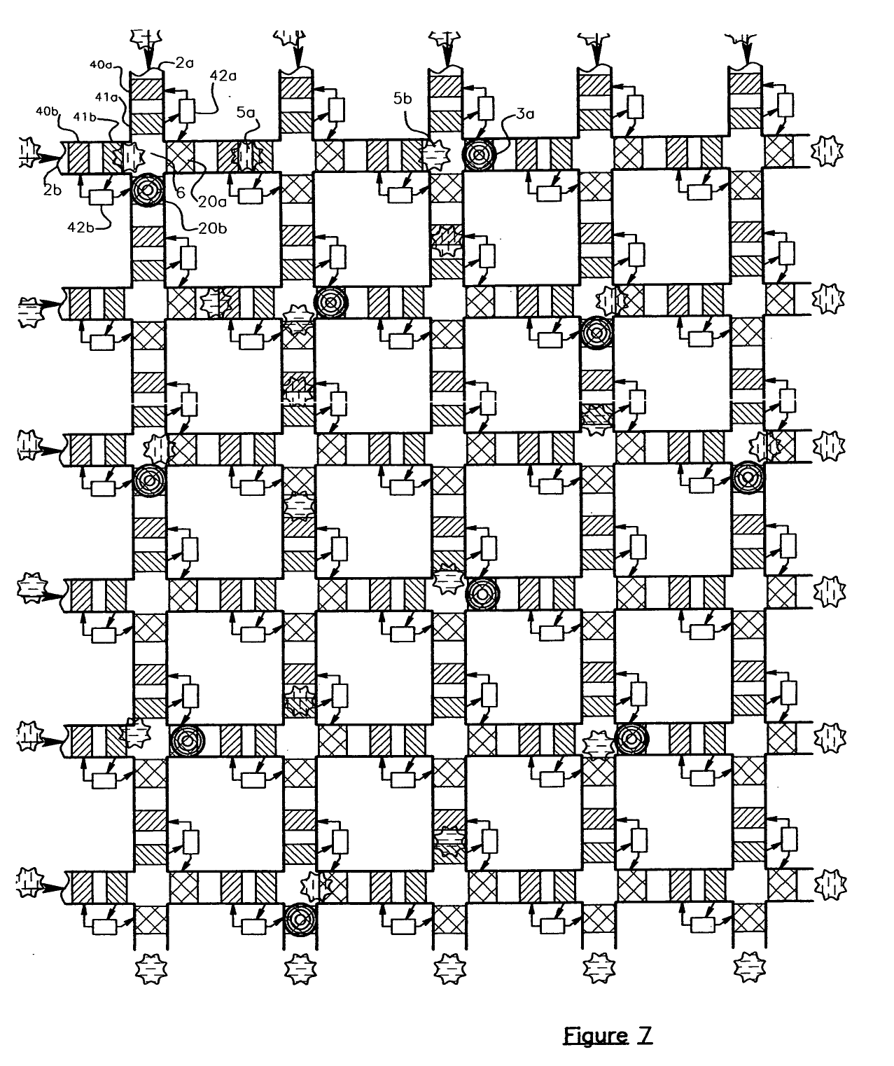

FIG. 7 shows a schematic view of a sequence of X-valves arranged in parallel and series. A multiplicity of entrances (4a) are generally disposed on the top edge of each junction (6). A multiplicity of entrances (4b) are generally disposed on the left of each junction. The exits (2a) and (2b) are generally disposed on the right and bottom of each junction respectively. Collectively, the array exits are to the right and bottom.

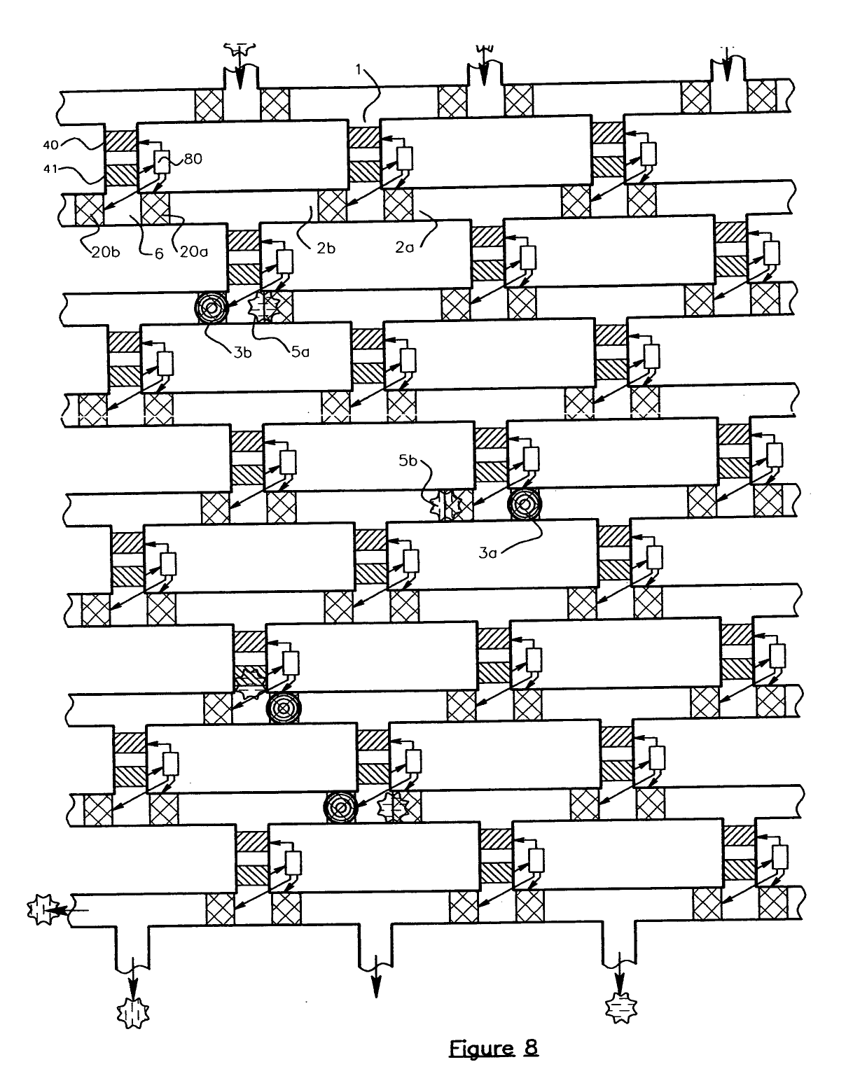

FIG. 8 shows a schematic view of a sequence of T-valves arranged in parallel offset rows. A multiplicity of entrances (4) are generally disposed on the top edge of the array. The exits (2a) and (2b) are disposed on the right and left of each junction (6). Collective exits are disposed at the bottom and or sides of the arrays. This arrangement has the additional benefit of one sensor group per junction.

As in earlier examples, discriminating sensors and bubble generating sites are disposed at many or all of the intersections. Sensors may be nominally identical. Sensors may have one variety in one direction and a second variety in the other direction. The sensors may have a wide variety throughout the structure.

Velocity of working fluid can be monitored and adjusted by the actuation of bubbles within channels.

Sensors do not need to be very efficient. The redundancy of multiple detectors gives the overall apparatus many chances to make decisions and correct errors in decisions.

The discrimination function may be achieved with external sensors. A natural choice is to use a CCD camera that can simultaneously visualize a large number of junctions. This would require communicating decisions to each of the bubble forming regions. This may be done through optical excitation of the bubbles through photo detectors. Alternately, control signals could be directed in to each of the resistors. Driver circuitry may be centralized or distributed.

An alternative method would be to do all of the sensing, discrimination, and driving locally at each junction. A data channel can be routed to each node for control functions. A data channel can also be provided to communicate out the details of the sort provided or the aggregate of the sort accomplished. Logic circuitry may also be centralized on the substrate.

Virtual walls can be formed by a series of bubbles. This greatly reduces the need for wall structures and the need to align wall structures with the structures on the substrate. If bubbles are generated by an externally focused laser or focused sound, particles could be deflected within a thick layer of working fluid.

Sorting can be arranged in a wide variety of configurations. These include but are not limited to the examples cited herein. The concentration of a population can be increased. One population can be separated from another. A continuum of properties can be sorted for presenting a distribution at the arrays of exit channels. A detailed sorting can be used to arrange components for chemical assembly at the exit ports.

The working fluid can be arranged in short or long segments separated by gas. So a sorting array can be used to move and direct fluids or gas products. Elastomeric layers can be used to isolate the working fluid from the fluid or gas being transported.

Particles, fluids and gasses can me manipulated by the switches to reaction sites where chemistry can be directed. Resulting components can then be detected, sorted, and or directed for further processing. Ink can be directed by bubble valves. This may be used to mix incoming colors and color densities of ink for subsequent delivery to ink jet nozzles.

1. A device for sorting cells and the like comprising:
a. one or more input channel(s)
b. a flow of suspended cells or particles,
c. two or more output channels,
d. a means for forming a vapor bubble occluding one or more of said output channels

2. a device as in claim 1 where said vapor bubble is formed by heat from a thin film resistor situated in said channels

3. a device as in claim 1 with a detection means for said cell

4. a device as in claim 4 where said cells are marked with a florescent dye

5. a device as in claim 5 where said detection means is a photo detector

6. a device as in claim 5 with control circuitry detecting signals from said photo detector(s) and drivers for said resistors.

7. A device for sorting cells and the like comprising:
a. one or more input channel(s),
b. two or more output channels,
c. a means for forming a vapor bubble occluding one or more of said output channels and,
d. a thin film resistor with control circuitry in close proximity to said channels

8. a device as in claim 7 where said cells are marked with a florescent dye

9. a device as in claim 8 where a photon generating device is situated in said input channel

10. a device as in claim 8 where a photon detector is situated in said input channel

11. a device as in claim 10 where said control circuitry is triggered to form said vapor bubble in response to detected photons

12. a device as in claim 11 where said control circuitry receives command controls from a data bus

13. a device as in claim 11 where said detection signals are relayed out through a data bus.

14. A method for sorting cells and the like comprising:
a. causing a flow of a suspension of cells through an input channel
b. detecting a particle in said input channel,
c. deciding which output channel to direct said particle, and
d. forming a vapor bubble to restrict said flow to one or more channels.

15. a method as in claim 14 in which said particles are marked with a florescent dye.

16. a method as in claim 15 in which said florescent dye is exposed to light while in said channel.

17. a method as in claim 16 where the light emitted by said fluorescing dye is detected by a photo detector.

18. a method as in claim 17 in which the detection of said light triggers the formation of said vapor bubble in one or more said channel(s).

19. a method as in claim 18 in which multiple input channels are processed in parallel

20. a method as in claim 19 in which multiple output channels present particles sorted by multiple markers
